(19) United States
(12) Patent Application Publication
(10) Pub. No.: US 2008/0057571 A1
(43) Pub. Date: Mar. 6, 2008

(54) BIOREACTOR DEVICE FOR EXPOSING A CELL CULTURE TO A FLUID SHEAR FORCE IMPARTED BY A FLUID FLOW

(75) Inventors: Elizabeth Loboa, Raleigh, NC (US); Nicholas Jardine, Cary, NC (US); Jennifer Jassawalla, Presto, PA (US); Jillian Rouse, Clinton, NC (US); Christopher Simms, Anchorage, AK (US); Jeffrey SooHoo, Apex, NC (US); J. Rebecca Stancil, Lillington, NC (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: North Carolina State University

(21) Appl. No.: 11/741,390

(22) Filed: Apr. 27, 2007

Related U.S. Application Data
(60) Provisional application No. 60/795,959, filed on Apr. 28, 2006.

(51) Int. Cl. C12M 3/00

(52) U.S. Cl. 435/293.1

(57) ABSTRACT

A system for exposing a cell culture to a fluid shear force imparted by a fluid flow is provided. Specifically, various embodiments of the present invention provide flow chambers defining channels for retaining a cell culture in the fluid flow wherein the flow chambers may be removably disposed within a bioreactor system such that the flow chambers may be removed and/or replaced without disturbing the cell cultures retained therein or disposed elsewhere within the bioreactor system. The flow chambers are composed of a transparent material such that a user of the system may observe the development of the cell culture retained within the chamber as the cell culture is imposed to a fluid shear stress imparted by the fluid flow.

### CROSS REFERENCE TO RELATED APPLICATION

This application claims priority from U.S. Provisional Application No. 60/795,959, filed Apr. 28, 2006, which is hereby incorporated by reference herein in its entirety.

### FIELD OF THE INVENTION

The various embodiments of the present invention relate generally to bioreactor devices for exposing a cell culture to a fluid shear force imparted by a fluid flow passing through a channel defined by a flow chamber.

### BACKGROUND OF THE INVENTION

Mesenchymal stem cells (MSCs) have the ability to differentiate into a variety of skeletal tissues and, as such, research is being conducted to optimize the growth and culture of MSCs for the regeneration and/or growth of skeletal tissues in a laboratory environment and/or in vivo for the repair and/or regeneration of damaged skeletal tissue. Such research focuses at least in part on the combination of chemical and mechanical factors that may influence and regulate the differentiation of human MSCs (hMSCs).

For example, in order to promote and/or evaluate osteogenic differentiation of MSCs, it is beneficial to apply a range of fluid shear stresses to MSCs growing in two-dimensional (2D) and three-dimensional (3D) MSC cultures while maintaining suitable temperature, carbon dioxide concentration, and adequate mass transport of nutrients through the cell culture for cell viability and differentiation. Cell culture in a fluid shear stress environment may also be useful for promoting muscle and/or liver cell differentiation.

Some conventional fluid shear bioreactors comprise chambers for applying a fluid shear force to a 2D cell culture (such as a cell culture disposed on a glass slide) by introducing a fluid flow adjacent to a substantially flat bottom portion and/or top portion of the bioreactor chamber where the glass slide may be placed. Such 2D fluid shear bioreactors rely largely on diffusion to achieve the mass transport of nutrients from the flow of fluid to the cells in the 2D cell culture. While such conventional 2D fluid shear bioreactors may be capable of achieving adequate mass transport rates and fluid shear forces to support a 2D cell culture, such cell cultures do not provide researchers an opportunity to observe cellular differentiation in a 3D cell culture, which is much more comparable to in vivo cell growth, development, and differentiation. Moreover, the diffusive mass transport achieved by conventional 2D fluid shear bioreactors may, in some cases, be insufficient for supporting the growth of a 3D cell culture (which may require the increased flow rates and shear forces produced by a perfusive laminar flow to achieve cell growth, development, and differentiation that approximates cell development in vivo).

Some 3D fluid shear bioreactors have been described in the research literature to address the shortcomings of the conventional 2D fluid shear bioreactors discussed herein. However, existing designs for 3D fluid shear bioreactors do not provide the capability to easily interchange various 3D cell cultures and/or 3D scaffolds used to support such cell cultures without having to completely disassemble the bioreactor assembly and therefore interrupt the progress of a research protocol that may involve assessing the effects of slight changes in fluid shear, flow rate, mass flow on hundreds of different 3D cell cultures. Furthermore, because conventional 3D fluid shear bioreactors require the complete and/or partial disassembly of the bioreactor system in order to remove and/or replace a particular cell culture (or scaffold supporting such a culture), the reassembly of such conventional 3D fluid shear bioreactors may introduce an unacceptable level of experimental uncertainty across various cell cultures that may be placed in the bioreactor chamber. For example, each disassembly and/or reassembly cycle of conventional 3D fluid shear bioreactors may introduce slight changes in flow pathways, flow rate, and/or other flow characteristics within the bioreactor that may compromise experimental results over a large population of cell cultures. Furthermore, existing 3D fluid shear bioreactors do not allow a user to visualize the 3D cell culture housed therein such that the user may visualize the effects of the fluid shear imposed by the bioreactor on the 3D cell culture in real time during the course of experimentation.

Thus, there exists a need in the art for a fluid shear bioreactor system that addresses the shortcomings of conventional 2D and 3D fluid shear bioreactors discussed herein. For example, there exists a need for a fluid shear bioreactor chamber (for retaining a cell culture therein) that may be easily removed and/or replaced within a fluid flow pathway without the need to completely and/or partially disassemble the flow pathways entering the chamber. In addition, there exists a need in the art for a fluid shear bioreactor that allows a user to easily visualize the effects of fluid shear forces that may be applied (via a 3D laminar flow of fluid, for example) to a cell culture that is retained within a flow chamber of the bioreactor. Furthermore, there exists a need for a fluid shear bioreactor that improves upon conventional 2D and 3D fluid shear bioreactors discussed in the scientific literature by providing a bioreactor that may be produced at a relatively low cost from easily-available modular components and that may facilitate the growth, development, and differentiation of cells within a large number of different cell cultures that may be evaluated and/or cultivated as part of a comprehensive research project and/or large-scale industrial process.

### SUMMARY OF THE INVENTION

The embodiments of the present invention satisfy the needs listed above and provide other advantages as described below. Embodiments of the present invention may include a system for exposing a three-dimensional cell culture to a fluid shear force imparted by a fluid flow. The system comprises a flow chamber adapted to be removably and serially engaged between an inlet tube and an outlet tube. For example, in some embodiments, the flow chamber may be removably and serially engaged between an inlet tube and an outlet tube via an interference fit. In some such embodiments, the flow chamber may comprise a substantially resilient material configured to receive the inlet tube and the outlet tube in an interference fit.

The flow chamber defines a channel extending therethrough, preferably in coaxial relation with the inlet tube and the outlet tube. Thus, the channel is configured to allow fluid communication between the inlet tube and the outlet tube such that the fluid flow is established upstream of the channel. The flow chamber is further adapted to retain as two- or three-dimensional cell culture within the channel such that the fluid flow applies a fluid shear force to the cell culture. Furthermore, the flow chamber is formed from a substantially transparent material such that a user of the system may visually observe the three-dimensional cell culture within the channel while the fluid flow applies the fluid shear force to the cell culture.

In some embodiments, the system comprises a plurality of flow chambers arranged in parallel and adapted to be removably and serially engaged between a corresponding plurality of inlet tubes and a corresponding plurality of outlet tubes. According to such embodiments, each of the plurality of flow chambers defines a channel extending therethrough in fluid communication with the corresponding one of the plurality of inlet tubes and the corresponding one of the plurality of outlet tubes such that the fluid flow is established upstream of the channel. In such embodiments, each of the plurality of flow chambers is configured for retaining one or more of a plurality of two- or three-dimensional cell cultures within the channel such that the fluid flow applies a fluid shear force to at least one cell culture. Furthermore, each of the plurality of flow chambers is formed from a substantially transparent material such that a user of the system may visually observe the at least one three-dimensional cell culture within the channel while the fluid flow applies the fluid shear force to the at least one cell culture.

According to some additional embodiments, the flow chamber comprises a proximal end in fluid communication with the inlet tube and a distal end in fluid communication with outlet tube. In addition, the proximal end and the distal end of the flow chamber may each comprise a disconnect device for removably engaging the flow chamber between the inlet tube and the outlet tube. According to some such embodiments, the disconnect may comprise a valve device for selectively preventing a fluid from entering or leaving the culture chamber and connecting tube such that the culture chamber may be removed and replaced while retaining fluid in the culture chamber and/or preventing fluid loss from the fluid source.

The system embodiments of the present invention may further comprise elements for retaining a three-dimensional cell culture within the channel defined in the flow chamber. For example, in some embodiments, the system further comprises a scaffold disposed within the channel of the flow chamber. The scaffold is configured to retain the three-dimensional cell culture and defines a plurality of apertures for allowing fluid communication between the inlet tube and the outlet tube while the three-dimensional cell culture is retained by the scaffold within the flow chamber. In some embodiments, the scaffold may comprise materials and/or structures that may include, but are not limited to: hydrogels; collagen scaffold complexes; polycaprolactone; polylactic acid; polyglycolic acid; polylactic-co-glycolic acid; polylactic-co-glycolic acid/polyethylene glycol block co-polymer; hydrogels; Type I collagen; Type II collagen; Type III collagen; Type IV collagen; laminin; fibronectin; agarose; alginate; and/or combinations of such materials and structures. According to some such embodiments, the system may further comprise a well device for removably retaining the scaffold within the channel of the flow chamber. The well device is configured to be removably disposed within the flow chamber such that the well device and the scaffold retained therein may be removed from and replaced in the flow chamber.

Various system embodiments may further comprise a pump device in fluid communication with the inlet tube for conveying a supply of fluid to the inlet tube for establishing the fluid flow within the channel. In some embodiments, the pump device comprises a pulsatile pump configured to convey the supply of fluid at a pulsed flow rate by exerting a pulsatile pumping action on the supply of fluid. Furthermore, some such embodiments may further comprise a fluid damping device removably engaged and in fluid communication between the pump device and the inlet tube. The fluid damping is configured to dampen the pulsatile pumping action such that the supply of fluid is conveyed to the inlet tube at a flow rate having an increased steadiness while the fluid damping device is in fluid communication between the pump device and the inlet tube.

Thus the various embodiments of the present invention provide many advantages that may include, but are not limited to: providing a bioreactor flow chamber capable of applying fluid shear stress to cell cultures that allows for easy, non-destructive removal and consistent placement of cell culture scaffolds; and providing a bioreactor system that comprises readily-available and inexpensive modular components that may be easily separated such that cell culture scaffolds may be inserted, removed, and observed while minimizing external forces and/or disturbances that may adversely affect the cell culture.

These advantages, and others that will be evident to those skilled in the art, are provided in the bioreactor system of the present invention.

### BRIEF DESCRIPTION OF THE DRAWINGS

Having thus described the invention in general terms, reference will now be made to the accompanying drawings, which are not necessarily drawn to scale, and wherein:

FIG. 1 shows a schematic of a flow chamber, according to one embodiment of the present invention, defining a flow channel for retaining a three dimensional cell culture;

FIG. 2 shows a schematic of a flow chamber, according to one embodiment of the present invention, defining a flow channel for retaining a three dimensional cell culture, and wherein the flow chamber includes quick disconnect devices for removably engaging the flow chamber between an inlet tube and an outlet tube;

FIG. 3 shows a schematic of a fluid shear bioreactor system, according to one embodiment of the present invention, comprising a plurality of flow chambers arranged in parallel and disposed within a rack;

FIG. 4 shows a schematic of a fluid shear bioreactor system, according to one embodiment of the present invention, further comprising a pump device for establishing a fluid flow from a fluid reservoir to an inlet tube and through a channel defined in a flow chamber;

FIG. 5 shows a schematic of a fluid shear bioreactor system, according to one embodiment of the present invention, wherein a flow chamber is disengaged from an outlet tube such that a well device for retaining a cell culture scaffold may be removed and/or replaced within one flow chamber while other parallel flow chambers remain in operation;

FIG. 6 shows a schematic of a fluid damping device, according to one embodiment of the present invention, that can be placed in fluid communication between a pump device and an inlet tube to dampen a pulsatile flow of fluid conveyed by the pump device; and

FIG. 7 shows an exploded schematic of a flow chamber, according to one embodiment of the present invention, defining a flow channel for retaining a two-dimensional cell culture in a recess adapted to receive a substantially flat slide.

### DETAILED DESCRIPTION OF THE INVENTION

The present inventions now will be described more fully hereinafter with reference to the accompanying drawings, in which some, but not all embodiments of the invention are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

While the various embodiments of the present invention are described herein in the context of a research environment for cultivating cell cultures and/or promoting cell differentiation of hMSCs (into a variety of cell types, such as skeletal tissue cells, cardiovascular tissue cells, and/or hepatic cells, for example) in a fluid-shear-inducing fluid flow, it should be understood that the various system embodiments described herein may also be used to expose various other cell cultures and/or scaffold structures to fluid shear forces in two-dimensional and/or three-dimensional arrangements. For example, the various system embodiments of the present invention may also be used to analyze the degradation of various scaffold types (wherein the scaffolds are used to retain a cell culture within a channel defined within a flow chamber through which the flow field extends) under varying magnitudes of fluid shear stress. Furthermore, and as described herein, various embodiments of the present invention may also be used to provide an easily-interchangeable and/or replaceable flow chamber formed from a substantially transparent material and having a substantially rectangular cross section suitable for exerting fluid shear forces on a 2D cell culture disposed on a flat plate or slide (see, for example, the 2D flow chamber 12d defining a recess 17 for retaining a flat glass slide (not shown) within a channel 11d, as shown in FIG. 7).

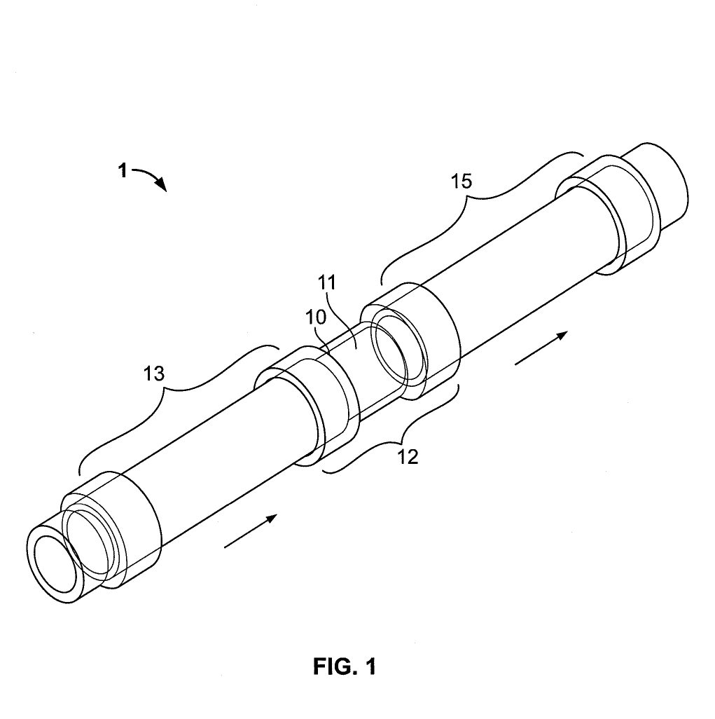

As shown in FIG. 1, some embodiments of the present invention may comprise a system 1 for exposing a three-dimensional cell culture (retained in a scaffold 10 (see FIG. 1, for example)) or a two-dimensional cell culture on a slide (see FIG. 7, for example) to a fluid shear force imparted by a fluid flow. The fluid may comprise a nutrient-rich and/or gas-infused media configured to promote the growth and/or development of the cells within the cell culture. In one embodiment, the system 1 comprises a flow chamber 12 adapted to be removably and serially engaged between an inlet tube 13 and an outlet tube 15. The flow chamber 12 defines a channel 11 extending therethrough in coaxial relation with the inlet tube 13 and the outlet tube 15 and the channel 11 is configured to allow fluid communication between the inlet tube 13 and the outlet tube 15 such that a fluid flow is established substantially upstream of the channel 11 (such that a laminar and/or turbulent fluid flow is realized before the fluid flow reaches a cell culture, scaffold 10 retained within the channel 11, and/or slide (see FIG. 7, for example).

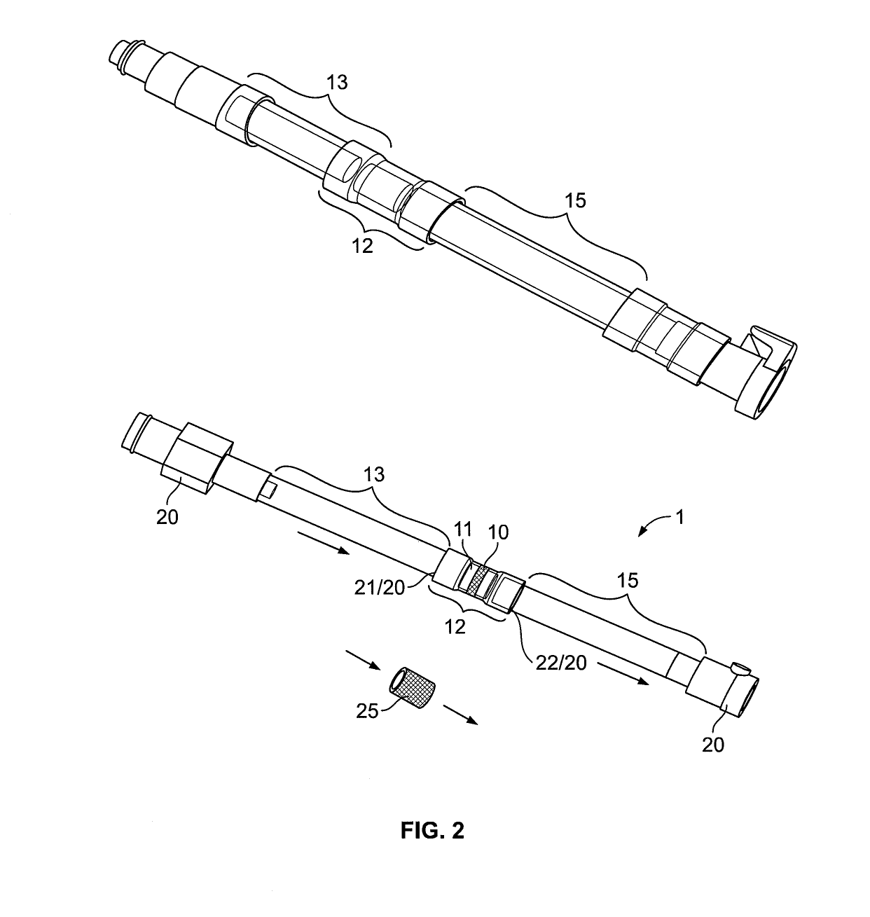

In some embodiments, the flow chamber 12, inlet and outlet tubes 13, 15 and other fluid-conveyance channels of the system 1 may be formed from tubing elements having substantially circular cross sections. In other embodiments, however, the flow chamber 12 and complementary tubing elements may also have cross sectional shapes that may include, but are not limited to: rectangles, ovals, and combinations of such shapes. For example, as shown generally in FIG. 7, some embodiments of the present invention may provide a flow chamber 12d defining a channel 11d having a substantially rectangular cross section, that may be configured to impart a fluid shear force on a 2D cell culture. For example, the 2D cell culture may be disposed on a substantially flat glass slide (not shown) that may be disposed within and retained by a recess 17 defined in a wall of the channel 11d. Thus, in some embodiments, the 2D cell culture housed on the slide may be disposed substantially parallel to a laminar flow field such that the fluid therein may nourish the cell culture (via diffusive flow, for example) and impart a fluid shear stress on the 2D cell culture. The 2D flow chamber 12d, shown in the embodiment of FIG. 7, may also provide many of the advantages described herein with respect to the 3D flow chamber 12 embodiments shown in FIGS. 1 and 2 (such as, for example, providing for real-time visualization of the cell culture, and the capability of removing and/or replacing individual cell cultures without the need to disassemble the entire bioreactor system 1). As described further herein with respect to the 3D flow chamber 12 shown in FIGS. 1 and 2, the 2D flow chamber 12d may also comprise one or more disconnect devices 20 for removably engaging the flow chamber 12d between an inlet tube and an outlet tube.

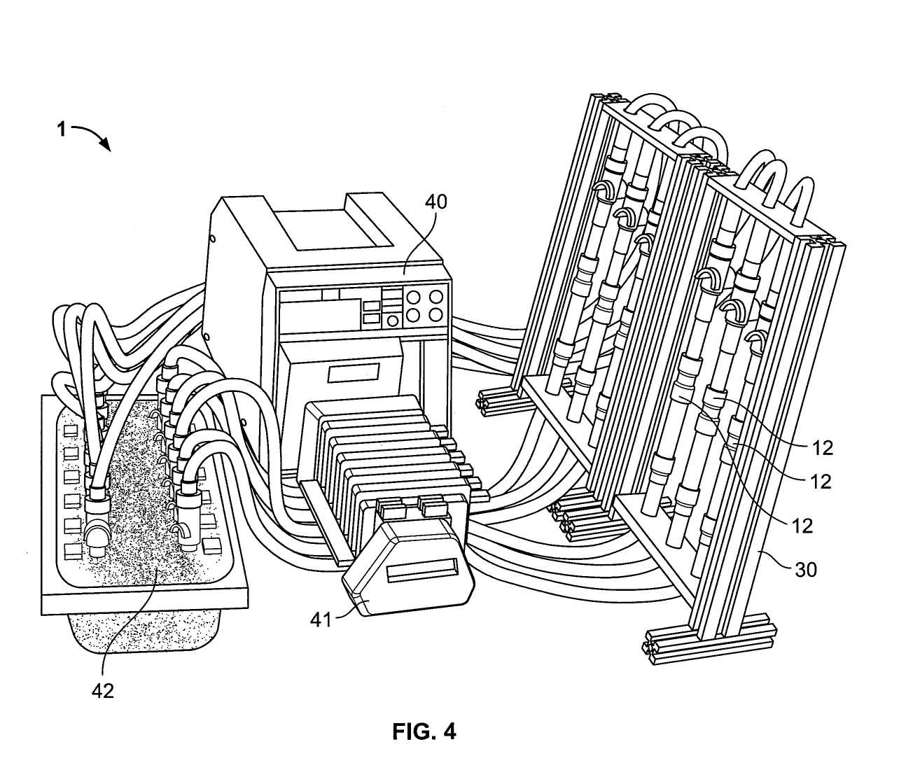

One skilled in the art will appreciate, for system 1 embodiments having flow chambers 12 that retain scaffolds 10 defining apertures with substantially circular cross sections, that fluid shear stress (τ) exerted on the cell culture retained in the channel 11 may be expressed according to the following relationship:
τ=8 μU/d (1)
where U is the average fluid velocity; t is the viscosity of the fluid conveyed through the system 1; and where d is a diameter of the apertures defined by the scaffold 10. Furthermore, the length of the inlet tube 13 and/or flow chamber 12 may be selected such that, as a fluid flow enters the inlet tube 13 (conveyed by a pump device 40, for example, as shown generally in FIG. 4), a laminar fluid flow is established over an entire cross-sectional area of the channel 11 defined within the flow chamber 12. Thus, according to the various system 1 embodiments of the present invention, a laminar perfusive fluid flow may be established upstream of the three-dimensional cell culture (i.e. prior to the flow reaching the cell culture) that is retained in the channel 11 of the flow chamber 12.

For example, in embodiments wherein the inlet tube 13, flow chamber 12, and outlet tube 15 are configured to have substantially circular cross-sections, the “entrance length” (L′) (of overall length) of the inlet tube 13 may be selected to ensure that “fully developed” laminar fluid flow is established upstream of the channel 11 defined in the flow chamber 12. For example, an approximate entrance length (L) of the inlet tube 13 may be calculated based upon a diameter (d) of the inlet tube 13 according to the following approximate relationship:
L≈(0.06)(ρUd²/μ) (2)
where ρ is the density of the fluid conveyed through the system 1; U is the average fluid velocity; and μ is the viscosity of the fluid conveyed through the system 1. Thus, using the relationships defined generally by equations (1) and (2), the system 1 embodiments of the present invention may be tailored to impart a precise fluid shear force on a 3D cell culture retained in a flow chamber 12 having a particular diameter using a fully-developed and/or substantially laminar fluid flow developed upstream of the flow chamber 12 (i.e., in an inlet tube 13).

In other embodiments, the length of the inlet tube may be tailored to develop a substantially turbulent fluid flow upstream of the flow chamber 12 (i.e., in an inlet tube 13) to model and/or replicate a plurality of in vivo vascular conditions. Such embodiments, may allow a researcher to view the development of the cell culture retained in the channel 11 in a substantially turbulent fluid flow field.

The flow chamber 12 is adapted to retain the cell culture within the channel 11 such that the fluid flow established within the system 1 applies a relatively constant fluid shear force to the cell culture. According to various embodiments of the present invention, the cell culture may be retained within the channel 11 by various types of scaffolds, media, and/or porous wells, as further described herein, that may be configured to have an outer dimension that approximates and/or exceeds an inner cross-sectional dimension of the channel such that the three-dimensional cell culture may be retained within the channel 11 via an interference fit. According to other embodiments, the flow chamber 12 may define one or more shoulders disposed along a wall of the channel 11 for retaining a scaffold 10 or other support structure for retaining the three-dimensional cell culture within the channel 11 defined by the flow chamber 12. As described herein with respect to FIG. 7, some system embodiments may further comprise a flow chamber 12d defining a recess 17 for receiving a flat glass slide for retaining a 2D cell culture adjacent and/or parallel to the channel 11d defined by the 2D flow chamber 12d.

Furthermore, the flow chamber 12 is formed from a substantially transparent material such that a user of the system 1 may visually observe the cell culture within the channel 11 while the fluid flow applies the fluid shear force to the cell culture. For example, the flow chamber 12 may, in some embodiments, be formed of a substantially transparent length of vinyl tubing (as shown, for example, in FIG. 2). Vinyl tubing may be ideal for use in forming the flow chamber 12 in some embodiments as the vinyl tubing may exhibit resilient and/or elastic mechanical properties such that a scaffold (and/or well device 25, as shown in FIG. 2) may be inserted into the channel 11 defined by the flow chamber 12 and retained therein via interference fit. In some embodiments, the flow chamber 12 may be formed of substantially transparent gas-permeable silicone tubing in order to promote oxygen and/or other gas exchange between the system 1 and the surrounding environment. The various 2D and 3D flow chambers 12, 12d described herein may also be formed of a variety of other substantially transparent materials that may include, but are not limited to: glass, silicone, vinyl, acrylic, polycarbonate, polymethyl methacrylate (PMMA), and combinations of such materials. As described herein, the flow chamber 12 and other system components (such as the inlet and outlet tubes 13, 15 and related tubing and fluid reservoirs 42 (see FIG. 4, for example)) may be formed from a selected biocompatible polymer compound suitable for use in regulatory-compliant industrial processes.

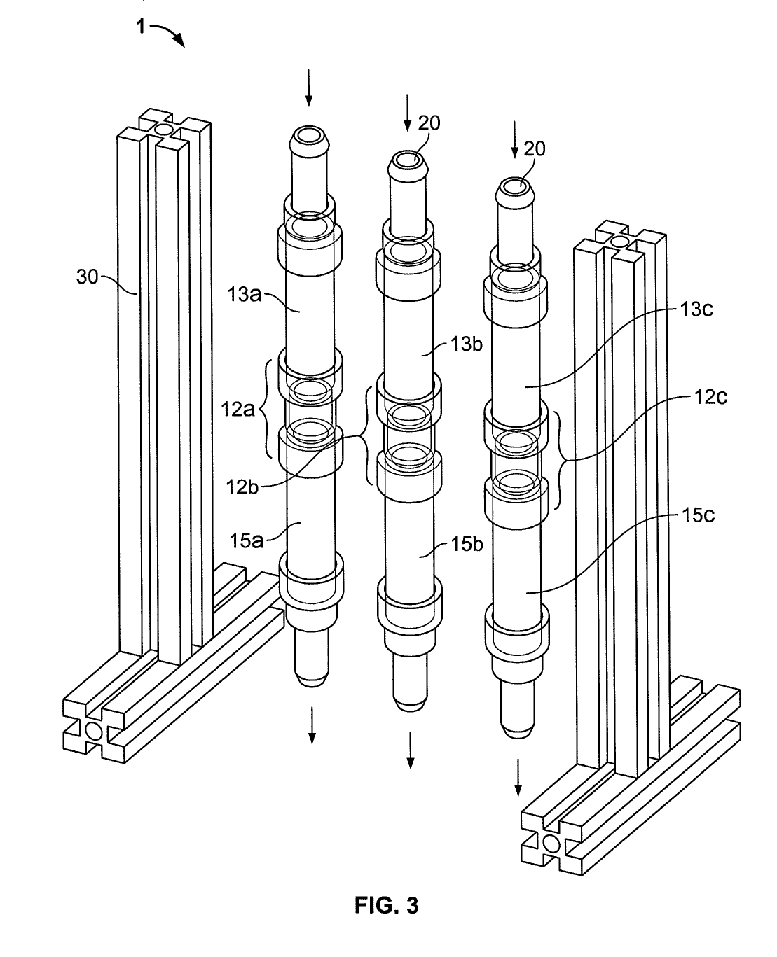

According to some system 1 embodiments, as shown generally in FIG. 2, the flow chamber 12 comprises a proximal end 21 in fluid communication with the inlet tube 13 and a distal end 22 in fluid communication with outlet tube 15. In some embodiments, the proximal end 21 and the distal end 22 of the flow chamber 12 each comprise a disconnect device 20 for removably engaging the flow chamber 12 between the inlet tube 13 and the outlet tube 15. As shown generally in FIG. 2, the disconnect device 20 may comprise a substantially resilient material (or a segment of such a material) operably engaged with the ends 21, 22 of the flow chamber. Thus, the resilient material may be elastically deformed such that the ends of the inlet and outlet tubes 13, 15 may be inserted into the ends 21, 22 of the flow chamber. For example, in some embodiments, the disconnect device 20 may comprise a section of silicone tubing material. According to other embodiments, the disconnect device 20 may comprise one or more commercially-available “quick-disconnect” devices for tubing that may include, but are not limited to: threaded luer-lock connectors, plug and socket tube fittings, compression tube fittings, barbed tube fittings, and combinations of such quick disconnect devices. The disconnect devices 20 may be formed of acetal or a selected biocompatible polymer compound for use in regulatory-compliant industrial processes. Furthermore, according to some embodiments, the disconnect device 20 may be operably engaged with the ends of the inlet and outlet tubes 13, 15 that are not operably engaged with the flow chamber 12 such that an entire system 1 subassembly (comprising, for example, the flow chamber 12, inlet and outlet tubes 13, 15, and connector devices 20) may be removably engaged between an inlet and outlet channel configured to convey a supply of fluid through the system 1 subassembly and/or flow chamber 12 (see FIG. 2, for example). Furthermore, as shown generally in FIG. 3, the system 1 embodiments of the present invention may also comprise a plurality of flow chambers 12a, 12b, 12c arranged in parallel and carried by a rack assembly 30. Each flow chamber 12a, 12b 12c may be removably engaged between corresponding inlet tubes (13a, 13b, 13c, respectively) and outlet tubes (15a, 15b, 15c, respectively). According to some embodiments, the disconnect devices 20 may further comprise corresponding valve devices for selectively preventing the fluid supply from entering the flow chamber 12 such that the flow chamber may be removed and replaced while retaining fluid in the channel 11 defined in the culture chamber 12 and/or preventing fluid loss from a fluid source (such as the fluid reservoir 42 shown in FIG. 4, for example).

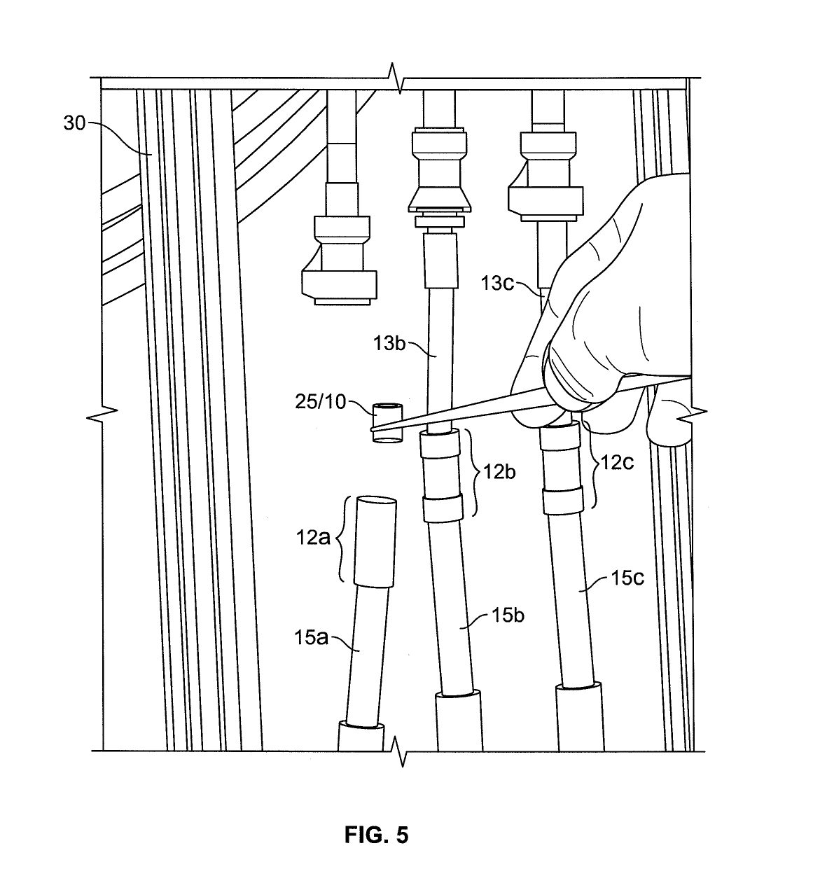

For example, the disconnect device 20 may be operably engaged between the inlet tube 13a and the flow chamber 12a such that as the flow chamber 12a is removed (such that a three-dimensional cell culture retained therein may be replaced, for example) a fluid flow through the first system 1 subassembly (13a, 12a, 15a, for example) may cease and the fluid flow (conveyed, for example, by a pump device 40, as shown in FIG. 4) may be diverted to one or more system 1 subassemblies (comprising, for example, flow chambers 12a and 12b) disposed in parallel with the first flow chamber 12a. According to other embodiments, as shown generally in FIG. 5, a scaffold 10 and/or well device 25 housing a cell culture may be removed and/or replaced within a first flow chamber 12a without disturbing the fluid flow (such as a laminar fluid flow field, for example) conveyed through one or more additional flow chambers 12b, 12c disposed in parallel with the first flow chamber 12a. For example, a valve device may be disposed upstream of the inlet tube 13a (as part of a disconnect device 20a configured to releasably engage the inlet tube 13a with the rack assembly 30, for example) for diverting a fluid flow from the first flow chamber 12a to one or more parallel flow chambers 12b, 12c within the system 1.

As shown in FIG. 2, some system 1 embodiments of the present invention may comprise a scaffold 10 disposed within the channel 11 of the flow chamber 12 for retaining the three-dimensional cell culture as the fluid flow exerts a fluid shear force on the cell culture. The scaffold 10 defines a plurality of apertures for allowing fluid communication between the inlet tube 13 and the outlet tube 15 while the three-dimensional cell culture is retained by the scaffold 10 within the flow chamber 12. The particular aperture geometry, material composition, and overall configuration of the scaffold 10 may be optimized for supporting various types of cell cultures. For example, in system embodiments wherein the cell culture comprises hMSC candidates for osteogenic differentiation, the scaffold 10 may comprise a relatively rigid mesh scaffold materials. In other embodiments, wherein the cell culture comprises candidates for hepatic cell differentiation, the scaffold 10 may comprise a relatively soft scaffold material such as a nonwoven fabric (including, but not limited to melt-spun nonwoven fabrics and hydroentangled nonwoven fabrics), a mesh fabric, a hydrogel, and/or a collagen complex. According to various embodiments of the present invention, the scaffold 10 may comprise one or more biodegradable and/or biocompatible materials that may include, but are not limited to: polycaprolactone; polylactic acid (PLA), polyglycolic acid (PGA), polylactic-co-glycolic acid (PLGA), PGLA/polyethylene glycol (PEG) block co-polymers, and combinations of such scaffold materials. In some embodiments, the scaffold 10 may also comprise a hydrogel or collagen-based scaffold material suitable for supporting hepatic cell cultures. Additional scaffold 10 materials may include, but are not limited to: hyaluronic hydrogels (optionally complexed with Type III or IV collagen and laminin); Type I-IV collagens; fibronectin; agarose; alginate; and/or combinations of such scaffold materials.

According to some embodiments, as shown generally in FIGS. 2 and 5, the system 1 may further comprise a well device 25 for removably retaining the scaffold 10 within the channel 11 of the flow chamber 12. Like the scaffold 10 itself, the well device 25 may be configured to be removably disposed within the flow chamber 12 such that the well device 25 and the scaffold 10 retained therein may be removed from and replaced in the flow chamber 12 (see, for example, FIG. 5). As shown in FIG. 2, the well device 25 may comprise a substantially cylindrical and/or frusto-conical “basket” including a floor member defining apertures for maintaining fluid communication between the inlet tube 13 and the outlet tube 13 via the channel 11 defined in the flow chamber 12. According to various system 1 embodiments, the well device 25 may comprise one or more polymeric, metallic, and/or metallic alloy materials suitable for removably retaining the scaffold 10 within the channel 11 defined in the flow chamber 12. System 1 embodiments comprising well devices 25 may be used to retain soft and/or pliant scaffolds 10 (such as hydrogels, for example) to prevent damage and/or excessive erosion of the scaffold 10 as it is suspended in the flow field within the channel 11.

As shown in FIG. 4, various system 1 embodiments of the present invention may also comprise a pump device 40 in fluid communication with the inlet tube 13 for conveying a supply of fluid to the flow chamber 12 (so as to establish a laminar fluid flow therein for imparting a fluid shear force, as described herein). The pump device 40 may also be configured to maintain mass transport through a cell-seeded scaffold 10 to promote the growth and/or differentiation of cells within a cell culture. The pump device 40 may comprise a plurality of pump channels 41 for individually conveying a supply of fluid (from a fluid reservoir 42, for example) to one or more of a plurality of flow chambers 12 (and associated inlet and outlet tubes 13, 15) disposed in parallel and carried by a rack assembly 30. The pump device 40 may comprise, in some embodiments, an electronically-controlled peristaltic and/or pulsatile pump configured to convey the supply of fluid at a selectable pulsed flow rate by exerting a pulsatile pumping action on the supply of fluid (drawn, for example, from a fluid reservoir 42, as shown generally in FIG. 4). According to other embodiments, the pump device 40 may comprise a variable speed pump device capable of conveying the supply of fluid to one or more inlet tubes 13 at a selectable and substantially constant flow rate. According to various embodiments, the pump device 40 may be electronically controlled to convey the supply of fluid to the inlet tube 13 at a flow rate that results in an optimal fluid velocity for establishing a laminar fluid flow within the flow chamber 12 (see Equation (2)) and a selected fluid shear stress (see Equation (1), for example) on the cell culture retained in the flow chamber 12. In some embodiments, each channel of the plurality of pump channels 41 (for individually conveying a supply of fluid from a fluid reservoir 42, for example) may be in fluid communication with a fluid reservoir 42 including a plurality of chambers that may be configured to supply at least one of the following: a particular fluid type for all the channels 41, a different particular fluid type for each channel of the plurality of channels 41; and/or supplying any combination of fluid types to each channel (and each downstream flow chamber 12).

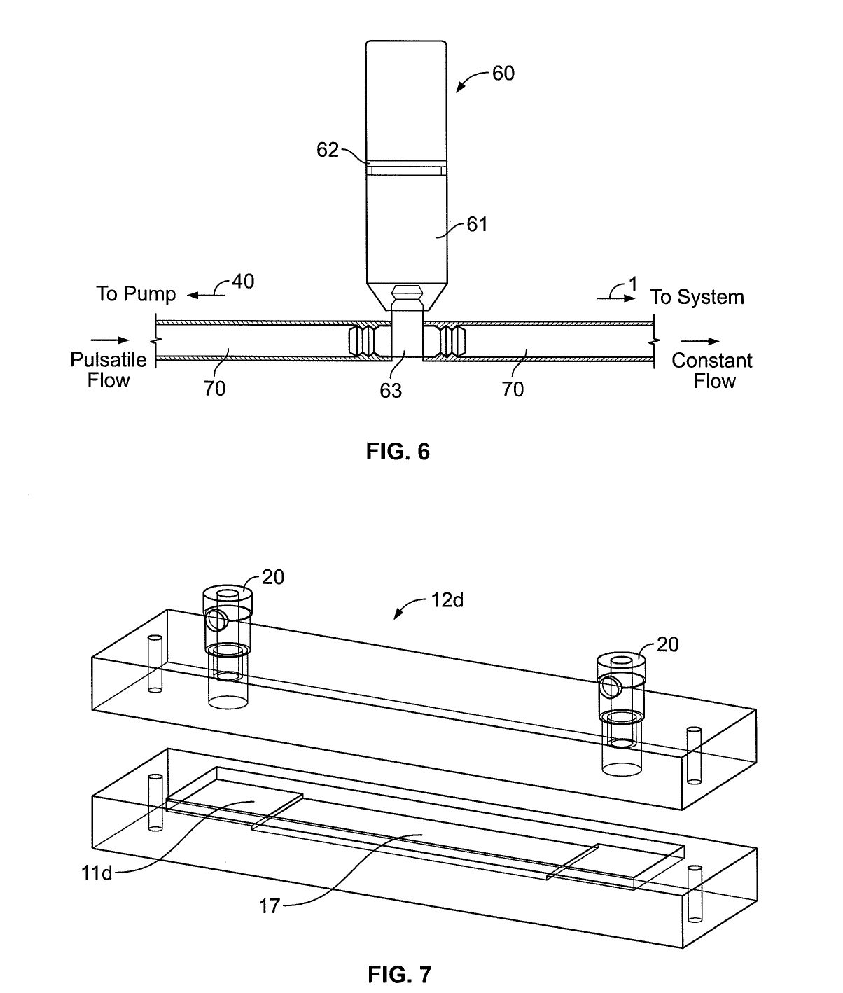

In some system embodiments, the system 1 shown generally in FIG. 4 may also comprise a fluid damping device 60 (see FIG. 6, for example) removably engaged and in fluid communication between the pump device 40 and the inlet tube 13 of the system 1. The fluid damping device 60 is configured to dampen the pulsatile pumping action such that the supply of fluid is conveyed to the inlet tube 13 (via an intermediate tube 70 for example) at a flow rate having an increased steadiness (and/or with less-pronounced pulsatile intensity “peaks”) while the fluid damping device 60 is in fluid communication between the pump device 40 and the inlet tube 13 (via a T-connector 63 or other tubing connection device). The damping device may comprise a cylindrical chamber 61 in fluid communication with a section of tubing 70 for establishing fluid communication between the pump device 40 and the inlet tube 13. Furthermore, as shown generally in FIG. 6, the cylindrical chamber 61 of the damping device may extend substantially perpendicularly to the direction of flow established by the section of tubing. Thus, the pulsatile pumping action of the pump device 40 may act to at least partially fill the damping device 60 with fluid such that the fluid interacts with a plunger device 62 slidably disposed within the cylindrical chamber 61 such that fluid exits the damping device 60 (and proceeds to the inlet tube 13) at a flow rate having an increased steadiness, regardless of the pulsatile pumping action of the pump device 40. Thus, such embodiments may allow a user of the system 1 to selectively insert the damping device 60 between the pump device 40 and the inlet tube 13 in order to selectively convert the fluid flow from a pulsatile flow to a substantially constant flow. Thus, the removal and/or engagement of the damping device 60 may allow a user of such systems 1 to evaluate the effects of pulsatile fluid flow (which may be considered analogous to in vivo conditions for cellular development) vs. substantially constant fluid flow, on the cell culture retained within the channel 11 of the flow chamber 12.

According to various other system 1 embodiments of the present invention, the various system components (such as the flow chambers 13 and fluid reservoir 42 shown generally in FIG. 4, for example) may be placed within an incubator device comprising an oxygen tension regulator for controlling and/or adjusting the oxygen and carbon dioxide conditions of the environment surrounding the system 1 and/or oxygen, carbon dioxide, or any other gases may be pumped directly into the media reservoir for increased gas concentration in the media. Furthermore, as shown in FIG. 4, some embodiments of the system 1 may comprise a rack assembly 30 carrying six flow chambers 12 disposed in parallel. It should be understood that any number of flow chambers 12 may be placed in parallel according to the various embodiments of the system 1 embodiments of the present invention (e.g. 2-20 flow chambers 12 in parallel and carried by a rack assembly 30). As described herein, the relative ease of interchangeability of the flow chambers 12 may allow a user to apply fluid shear stress to a variety of different 3D and/or 2D cell cultures retained within specialized flow chambers 12, 12d.

The scale of the system embodiments described herein may also be selected to suit various research and/or industrial applications. The chambers 12, 12d and/or inlet and outlet 13, 15 tubes may be sized, in some embodiments, to perform generally macrofluidic processes. Furthermore, according to some embodiments, the components of the system may also be reduced in scale to accommodate generally microfluidic processes. Various flow chambers 12 and/or inlet and outlet 13, 15 tubes may be provided to accommodate 3D cell cultures (or scaffolds 10 retaining such cell cultures) having a cross-sectional diameter ranging from very small fractions of an inch (such as 1/8″ (0.32 cm) or less) up to diameters of or exceeding 1 inch (2.54 cm). The 2D flow chamber 12d embodiments disclosed herein (as shown in FIG. 7, for example) may also define recesses 17 for receiving and retaining substantially flat slides having a variety of sizes, such as 25 mm by 75 mm by 1.2 mm “standard” slides, and/or more specialized slide sizes (such as, for example, 38 mm by 75 mm by 1.2 mm slides) configured to house 2D cell cultures.

Furthermore, the pump device 40 (as described herein) may comprise six corresponding flow channels 41 for supplying synchronous flow to each of the six flow chambers 12 shown generally in FIG. 4. The pump device 40 (and inlet tubes 13, in fluid communication therewith) may be configured to convey the supply of fluid from a fluid reservoir 42 to the flow chamber 12 at a flow rate that may be varied, for example, from about 0.4 milliliters/minute to about 28 milliliters/minute to achieve a selected shear stress (τ, as defined, for example in Equation (1) herein) that may be applied to the cell culture.

Furthermore, the tubing elements used to establish fluid communication between the fluid reservoir 42 and the pump device 40, and between the channels 41 of the pump device 40 and the inlet tubes 13 may comprise, for example, gas-permeable silicone tubing having an inner diameter of approximately ¼ inch (0.64 cm) to encourage gas exchange between the system 1 and the surrounding environment (which, as discussed herein, may be closely controlled by placing the system 1 in a cleanroom and/or incubator device).

While various embodiments of the invention described herein (as shown in FIG. 3, for example) include a plurality of flow chambers 12a, 12b, 12c arranged in parallel and retained in a rack assembly 30, it should be understood that the flow chambers 12, 12d of the present invention may also be arranged in series, such that the outlet tube 15 of a first flow chamber 12 is in fluid communication with an inlet tube 13 of a second flow chamber 12 (positioned “downstream” of the first) to form a single fluid flow path that may advance through: (1) a fluid reservoir 42, (2) a pump device 40 (and/or a fluid channel 41 thereof), (3) a series of flow chambers 12 (and complementary inlet and outlet tubes 13, 15 and, (4) to a fluid waste receptacle. Furthermore, according to various embodiments, the fluid may be filtered, treated, and/or reclaimed such that the fluid may be recycled through the parallel system 1 shown generally in FIG. 4 (or through a similar system wherein the flow chambers 12 are disposed in series).

Additionally, although not shown in the figures, the system 1 can further include means for introducing various compositions upstream of the cell culture in order to, for example, induce a desired cellular response. Exemplary compositions include various factors (e.g., growth factors) and pharmaceuticals. Such compounds or compositions can be introduced into the media stream upstream of the cell culture prior to the inlet of the flow chamber 12 using a variety of techniques. For example, where flexible tubing material is used upstream of the flow chamber 12, a composition can be simply injected into the media flow by puncturing the wall of the tubing with a needle. Alternatively, a valved intersection can be created upstream of the flow chamber 12, where a composition can be introduced into the media stream by action of the valve when desired. Likewise, downstream sampling of media after interaction with the cell culture can occur in the system 1 by addition of a sampling port, such as a valved port, downstream of the flow chamber 12. Alternatively, the media flowing through the system 1 can be collected in a container, rather than recycled in a recirculating manner, and sampled as desired. The ability to sample media downstream of the cell culture can aid, for example, the investigation of the effects of certain factors (added upstream) on the cultured cells.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A system for exposing a cell culture to a fluid shear force imparted by a fluid flow, the system comprising a flow chamber adapted to be removably and serially engaged between an inlet tube and an outlet tube, the flow chamber defining a channel extending therethrough in fluid communication with the inlet tube and the outlet tube such that the fluid flow is established upstream of the channel;
the flow chamber being further adapted to retain the cell culture within the channel such that the fluid flow applies a fluid shear force to the cell culture; and
the flow chamber being formed from a substantially transparent material such that a user of the system may visually observe the cell culture within the channel while the fluid flow applies the fluid shear force to the cell culture.

2. A system according to claim 1, wherein the flow chamber comprises a proximal end in fluid communication with the inlet tube and a distal end in fluid communication with outlet tube and wherein the proximal end and the distal end of the flow chamber each comprise a disconnect device for removably engaging the flow chamber between the inlet tube and the outlet tube.

3. A system according to claim 2, wherein the disconnect comprises a valve device for selectively preventing a fluid from entering or exiting the flow chamber such that the flow chamber may be removed and replaced while retaining fluid in the channel defined in the flow chamber.

4. A system according to claim 1, further comprising a scaffold disposed within the channel of the flow chamber configured to retain the cell culture, the scaffold defining a plurality of apertures for allowing fluid communication between the inlet tube and the outlet tube while the cell culture is retained by the scaffold within the flow chamber.

5. A system according to claim 1, further comprising a hydrogel disposed within the channel of the flow chamber configured to retain the cell culture, the hydrogel configured for allowing fluid communication between the inlet tube and the outlet tube while the cell culture is retained by the hydrogel within the flow chamber.

6. A system according to claim 1, further comprising a collagen scaffold complex disposed within the channel of the flow chamber configured to retain the cell culture, the collagen scaffold complex configured for allowing fluid communication between the inlet tube and the outlet tube while the cell culture is retained by the collagen scaffold complex within the flow chamber.

7. A system according to claim 4, wherein the scaffold comprises a material selected from the group consisting of:
polycaprolactone;
polylactic acid;
polyglycolic acid;
polylactic-co-glycolic acid;
polylactic-co-glycolic acid/polyethylene glycol block co-polymer;
hydrogels;
Type I collagen;
Type II collagen;
Type III collagen;
Type IV collagen;
laminin;
fibronectin;
agarose;
alginate; and
combinations thereof.

8. A system according to claim 4, further comprising a well device for removably retaining the scaffold within the channel of the flow chamber, the well device being configured to be removably disposed within the flow chamber such that the well device and the scaffold retained therein may be removed from and replaced in the flow chamber.

9. A system according to claim 1, further comprising a pump device in fluid communication with the inlet tube, the pump device being configured to convey a supply of fluid to the inlet tube for establishing the fluid flow within the channel.

10. A system according to claim 9, wherein the pump device comprises a pulsatile pump configured to convey the supply of fluid at a pulsed flow rate by exerting a pulsatile pumping action on the supply of fluid.

11. A system according to claim 10, further comprising a fluid damping device removably engaged and in fluid communication between the pump device and the inlet tube, the fluid damping device being configured to dampen the pulsatile pumping action such that the supply of fluid is conveyed to the inlet tube at a flow rate having an increased steadiness while the fluid damping device is in fluid communication between the pump device and the inlet tube.

12. A system according to claim 1, wherein the flow chamber is configured to retain the cell culture selected from the group consisting of:
a three-dimensional cell culture disposed on a scaffold disposed substantially perpendicularly to a flow direction the fluid flow;
a two-dimensional cell culture disposed on a substantially flat slide disposed substantially parallel to a flow direction of the fluid flow; and
combinations thereof.

13. A system according to claim 1, wherein the flow chamber is configured to retain a cell culture selected from the group consisting of:
a cell culture comprising hepatic cells;
a cell culture comprising cardiovascular tissue cells;
a cell culture comprising mesenchymal stem cells;
a cell culture comprising osteogenic cells derived from stem cells; and
a cell culture comprising muscle cells derived from stem cells.

14. A system according to claim 1, wherein the fluid flow within the channel comprises a laminar fluid flow.

15. A system for exposing a plurality of cell cultures to a fluid shear force imparted by a fluid flow, the system comprising a plurality of flow chambers arranged in parallel and adapted to be removably and serially engaged between a corresponding plurality of inlet tubes and a corresponding plurality of outlet tubes, each of the plurality of flow chambers defining a channel extending therethrough in fluid communication with the corresponding one of the plurality of inlet tubes and the corresponding one of the plurality of outlet tubes such that the fluid flow is established upstream of the channel;
each of the plurality of flow chambers being further adapted to retain at least one of the plurality of cell cultures within the channel such that the fluid flow applies a fluid shear force to the at least one of the plurality of cell cultures; and
each of the plurality of flow chamber being formed from a substantially transparent material such that a user of the system may visually observe at least one of the plurality of cell cultures within the channel while the fluid flow applies the fluid shear force to the at least one of the plurality of cell cultures.

16. A system according to claim 15, wherein the fluid flow within the channel comprises a laminar fluid flow.

17. A system for exposing a cell culture to a fluid shear force imparted by a fluid flow, the system comprising:
an inlet tube;
an outlet tube; and
a flow chamber configured to be removably and serially engaged with an interference fit between the inlet tube and the outlet tube, the flow chamber defining a channel extending therethrough in fluid communication with the inlet tube and the outlet tube such that the fluid flow is established upstream of the channel, the flow chamber being further configured to retain the cell culture within the channel such that the fluid flow applies a fluid shear force to the cell culture, the flow chamber being formed from a substantially transparent material such that a user of the system may visually observe the cell culture within the channel while the fluid flow applies the fluid shear force to the cell culture; and
a scaffold disposed within the channel of the flow chamber configured to retain the cell culture, the scaffold being configured for allowing fluid communication between the inlet tube and the outlet tube while the cell culture is retained by the scaffold within the flow chamber.

18. A system according to claim 17, wherein the flow chamber comprises a resilient material, and wherein the flow chamber is configured for receiving the inlet tube and the outlet tube.

19. A system according to claim 17, wherein the scaffold comprises a hydrogel disposed within the channel of the flow chamber configured to retain the cell culture, the hydrogel configured for allowing fluid communication between the inlet tube and the outlet tube while the cell culture is retained by the hydrogel within the flow chamber.

20. A system according to claim 17, wherein the scaffold a collagen scaffold complex disposed within the channel of the flow chamber configured to retain the cell culture, the collagen scaffold complex configured for allowing fluid communication between the inlet tube and the outlet tube while the cell culture is retained by the collagen scaffold complex within the flow chamber.

21. A system according to claim 17, wherein the scaffold comprises a material selected from the group consisting of:
polycaprolactone;
polylactic acid;
polyglycolic acid;
polylactic-co-glycolic acid;
polylactic-co-glycolic acid /polyethylene glycol block co-polymer;
hydrogels;
Type I collagen;
Type II collagen;
Type III collagen;
Type IV collagen;
laminin;
fibronectin;
agarose;
alginate; and
combinations thereof.

22. A system according to claim 17, further comprising a well device for removably retaining the scaffold within the channel of the flow chamber, the well device being configured to be removably disposed within the flow chamber such that the well device and the scaffold retained therein may be removed from and replaced in the flow chamber.

23. A system according to claim 17, wherein the fluid flow within the channel comprises a laminar fluid flow.
